SEQUENCE LISTING (9 sequences)

SEQ ID NO: 1 - DNA, 20 bp, Artificial
  primer
cccgtctacctctctccttg

SEQ ID NO: 2 - DNA, 20 bp, Artificial
  primer
gagcaagtgctgagggttta

SEQ ID NO: 3 - DNA, 24 bp, Artificial
  primer
cctggaacagcgctactgatcacc

SEQ ID NO: 4 - DNA, 20 bp, Artificial
  primer
agatcctgggtgcagacttc

SEQ ID NO: 5 - DNA, 20 bp, Artificial
  primer
aagggagcggtcagagaata

SEQ ID NO: 6 - DNA, 24 bp, Artificial
  primer
cgtcctcaaaggtctcctggaacc

SEQ ID NO: 7 - DNA, 20 bp, Artificial
  primer
cccccactgaaaaagatgag

SEQ ID NO: 8 - DNA, 18 bp, Artificial
  primer
atccaatccaaatgcggc

SEQ ID NO: 9 - DNA, 25 bp, Artificial
  primer
cctgccgtgtgaaccatgtgacttt

(19) United States
(12) Patent Application Publication
(10) Pub. No.: US 2011/0251220 A1
(43) Pub. Date: Oct. 13, 2011

(54) Pharmaceutical compositions comprising gamma secretase modulators

(76) Inventors: Özlem Acikgöz, Berlin (DE); Jessica Ahmed, Berlin (DE); Bernd Dörken, Berlin (DE); Cornelius Frömmel, Gottingen (DE); Andrean Goede, Berlin (DE); Franziska Jundt, Berlin (DE); Rudolf Kunze, Flieth-Stegelitz (DE); Robert Preissner, Berlin (DE)

(21) Appl. No.: 12/995,741

(22) PCT Filed: Jun. 2, 2009
(86) PCT No.: PCT/EP09/03915

§ 371 Date: Jun. 20, 2011

(30) Foreign Application Priority Data
Jun. 3, 2008 (EP) 08010126.4

(51) Int. Cl. A61K 31/513; C07D 239/56; A61K 31/4412; A61K 31/44; A61P 35/00; A61P 35/02; A61P 25/28; A61P 25/00; A61P 9/10; A61P 27/02; A61P 13/12; A61P 13/08; A61P 1/18; A61P 1/16; C07D 213/76

(52) U.S. Cl. 514/274; 546/297; 544/312; 514/349

(57) ABSTRACT

The present invention relates to pharmaceutical compositions comprising gamma secretase modulators as well as to the use of gamma secretase modulators for treating renal disorders, cancer, neurodegenerative disorders as well as related disorders.

The present invention relates to pharmaceutical compositions comprising gamma secretase modulators as well as to the use of gamma secretase modulators for treating renal disorders, cancer, neurodegenerative disorders as well as related disorders.

The renal glomerulus is responsible for ultrafiltration of the blood and ensures that essential plasma proteins are retained. Dysfunctions of the podocytes, which are the glomerular filter cells involved in the ultrafiltration process, play an important role in the development of renal diseases that afflict a growing number of patients.

The Notch signaling pathway comprises a family of transmembrane receptors. In humans there are four Notch receptors and five ligands (Jagged family and Delta family of Notch ligands). Binding of a ligand renders the Notch receptor susceptible to metalloprotease- and gamma-secretase-mediated proteolytic cleavage. The Notch pathway is crucial in podocyte development. Activating the Notch pathway in podocytes induces podocyte loss and glomerular failure. Gamma secretase inhibitors can prevent disease onset in a toxic podocyte damage model and are also beneficial as therapeutic agents in established glomerular filtration barrier faliure [T. Niranjan et al, The Noth pathway in podocytes plays a role in the development of glomerular disease. Nature Medicine, Volume 14, number 3, pages 290-298, March 2008; M. Kretzler and L. Allred, Notch inhibition reverses kidney failure. Nature Medicine, Volume 14, number 3, pages 246-247].

Furthermore, the Notch signaling pathway also plays a role in multiple myeloma (MM) cell growth and inhibition of Notch signaling with gamma secretase inhibitors presents a tool for downregulating Notch activity and suppressing MM cell growth [Shih le M, Wang T L. Notch signaling, gamma-secretase inhibitors, and cancer therapy. Cancer Res. 2007; 67: 1879-1882].

Gamma secretase inhibitors are also useful in the treatment of neurodegenerative disorders such as Morbus Alzheimer [U.S. Pat. No. 6,756,511; U.S. Pat. No. 6,683,091].

Accordingly, it was an object of the present invention to provide novel pharmaceutical compositions suitable for modifying the activity of gamma secretase and which may therefore be used for the treatment of disorders that are at least partially effected via gamma secretase activity such as renal disorders, cancer and neurodegenerative disorders.

Said object has been achieved by the provision of pharmaceutical compositions according to the present invention. It was surprisingly found that the compounds present in the inventive pharmaceutical compositions are useful as modulators of gamma secretase. In particular these compounds have an activity as inhibitors of gamma secretase.

Thus, according to one of its aspects, the present invention relates to a pharmaceutical composition comprising one or more compounds of general formula I or a pharmaceutically acceptable salt thereof:

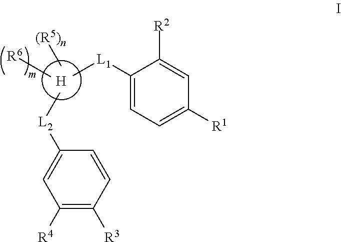

wherein
n, m mutually independent, each represent 0 or 1;

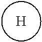

represents a 5, 6 or 7 membered carbocycle, wherein said carbocycle is partially unsaturated or aromatic and wherein said carbocycle contains 0, 1 or 2 nitrogen atoms as ring members;
L¹ is selected from the group consisting of C₁₋₆-alkylen; C(═O); C(═O)—N(H); N(H)—C(═O); N(H)—S(═O)₂ and S(═O)₂—N(H);
L² is selected from the group consisting of S; O; C₁₋₆ alkylen-C(═O); S—C₁₋₆ alkylen-C(═O); O—C₁₋₆ alkylen-C(═O); C(═O)—C₁₋₆-alkylen; C(═O)—C₁₋₆-alkylen-S; C(═O)—C₁₋₆-alkylen-O; C₁₋₆-alkylen-C(═O)—N(H); S—C₁₋₆-alkylen-C(═O)—N(H); O—C₁₋₆-alkylen-C(═O)—N(H); C(═O)—N(H)—C₁₋₆-alkylen; C(═O)—N(H)—C₁₋₆-alkylen-S; C(═O)—N(H)—C₁₋₆-alkylen-O; C₁₋₆-alkylen-N(H)—C(═O); S—C₁₋₆-alkylen-N(H)—C(═O); O—C₁₋₆-alkylen-N(H)—C(═O); N(H)—C(═O)—C₁₋₆ alkylen; N(H)—C(═O)—C₁₋₆ alkylen-S; N(H)—C(═O)—C₁₋₆ alkylen-O; C₁₋₆-alkylen-N(H)—S(═O)₂; S—C₁₋₆-alkylen-N(H)—S(═O)₂; O—C₁₋₆-alkylen-N(H)—S(═O)₂; N(H)—S(═O)₂—C₁₋₆-alkylen; N(H)—S(═O)₂—C₁₋₆-alkylen-S; N(H)—S(═O)₂—C₁₋₆-alkylen-O; C₁₋₆-alkylen-S(═O)₂—N(H); S—C₁₋₆-alkylen-S(═O)₂—N(H); O—C₁₋₆-alkylen-S(═O)₂—N(H); S(═O)₂—N(H)—C₁₋₆-alkylen; S(═O)₂—N(H)—C₁₋₆-alkylen-S; S(═O)₂—N(H)—C₁₋₆-alkylen-O;
   with the proviso that L¹ and L² are linked to vicinal ring-members of the carbocycle;
R¹, R² are each selected from the group consisting of —H; halogen; C₁₋₆ alkyl; C₂₋₆ alkenyl; C₂₋₆ alkynyl; —O—C₁₋₆ alkyl; —S—C₁₋₆ alkyl; C₁₋₆-haloalkyl; —O—C₁₋₆ haloalkyl; —S—C₁₋₆-haloalkyl; —OH; —SH; —CN; —NO₂ and —NR^{a}R^{b}, wherein R^{a} and R^{b} are independently H or C₁₋₆ alkyl;
   with the proviso that at least one of R¹ and R² represents H;
R³, R⁴ are each selected from the group consisting of —H; halogen; C₁₋₆ alkyl; C₂₋₆ alkenyl; C₂₋₆ alkynyl; —O—C₁₋₆ alkyl; —S—C₁₋₆ alkyl; C₁₋₆-haloalkyl; —O—C₁₋₆ haloalkyl; —S—C₁₋₆-haloalkyl; —OH; —SH; —CN; —NO₂ and —NR^{a}R^{b}, wherein R^{a} and R^{b} are independently H or C₁₋₆ alkyl;
   with the proviso that at least one of R³ and R⁴ does not represent H;
R⁵, R⁶ mutually independent, are each selected from the group consisting of —H; ═O; halogen; C₁₋₆ alkyl; C₂₋₆ alkenyl; C₂₋₆ alkynyl; —O—C₁₋₆ alkyl; —S—C₁₋₆ alkyl; C₁₋₆-haloalkyl; —O—C₁₋₆ haloalkyl; —S—C₁₋₆-haloalkyl; —OH; —SH; —CN; —NO₂ and —NR^{a}R^{b}, wherein R^{a} and R^{b} are independently H or C₁₋₆ alkyl.

According to the present invention the term halogen denotes —F, —Cl, —Br and —I, preferably —F, —Cl and —Br, yet more preferably —F and —Cl.

As used herein the expression C₁₋₆-alkyl represents linear or branched, saturated carbon chains having 1, 2, 3, 4, 5 or 6 carbon atoms. Examples of such alkyl moieties are methyl; ethyl; n-propyl; iso-propyl; n-butyl; iso-butyl; sec-butyl; tert-butyl; n-pentyl; iso-pentyl; neo-pentyl and hexyl.

As used herein the expression C₂₋₆-alkenyl denotes linear or branched, unsaturated carbon chains having 2, 3, 4, 5 or 6 carbon atoms. Said alkenyl moities have at least one C═C-double bond. Examples of such alkenyl moities are vinyl, prop-1-enyl and allyl.

As used herein the expression C₂₋₆-alkynyl denotes linear or branched, unsaturated carbon chains having 2, 3, 4, 5 or 6 carbon atoms. Said alkynyl moities have at least one C≡C-triple bond. Examples of such alkynyl moities are —C≡C— and —C≡C—CH₃.

As used herein the expression C₁₋₆-haloalkyl represents linear or branched, saturated carbon chains having 1, 2, 3, 4, 5 or 6 carbon atoms that are substituted with one or more, e.g. 1, 2, 3, 4 or 5, halogen atoms that may be identical or different. Examples of such haloalkyl moieties are —CF₃ and —CF₂—CF₃.

The expression C₁₋₆-alkylen as used herein denotes linear or branched, saturated carbon chains that link two moities. Examples of such alkylen moities are —CH₂—, —CH₂—CH₂—, —CH(CH₃)—, —CH₂—CH₂—CH₂—, —CH(CH₃)—CH₂—, —CH(CH₂CH₃)—, —CH₂—(CH₂)₂—CH₂—, —CH(CH₃)—CH₂—CH₂—, —CH₂—CH(CH₃)—CH₂—, —CH(CH₃)—CH(CH₃)—, —CH(CH₂CH₃)—CH₂—, —C(CH₃)₂—CH₂—, —CH(CH₂CH₂CH₃)—, —C(CH₃)(CH₂CH₃)—, —CH₂—(CH₂)₃—CH₂—, —CH(CH₃)—CH₂—CH₂—CH₂—, —CH₂—CH(CH₃)—CH₂—CH₂—, —CH(CH₃)—CH₂—CH(CH₃)—, —CH(CH₃)—CH(CH₃)—CH₂—, —C(CH₃)₂—CH₂—CH₂—, —CH₂—C(CH₃)₂—CH₂—, —CH(CH₂CH₃)—CH₂—CH₂—, —CH₂—CH(CH₂CH₃)—CH₂—, —C(CH₃)₂—CH(CH₃)—, —CH(CH₂CH₃)—CH(CH₃)—, —C(CH₃)(CH₂CH₃)—CH₂—, —CH(CH₂CH₂CH₃)—CH₂—, —C(CH₂CH₂CH₃)—CH₂—, —CH(CH₂CH₂CH₂CH₃)—, —C(CH₃)(CH₂CH₂CH₃)—, —C(CH₂CH₃)₂— and —CH₂—(CH₂)₄—CH₂—. Examples of such alkylen moities that may typically be preferred are —CH₂—, —CH₂—CH₂— and —CH₂—CH₂—CH₂—.

In another one of its aspects, the present invention relates to a pharmaceutical composition comprising one or more compounds of general formula I or a pharmaceutically acceptable salt thereof:

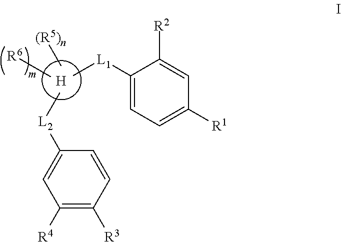

wherein
n, m mutually independent, each represent 0 or 1;

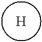

represents a 5, 6 or 7 membered carbocycle, wherein said carbocycle is partially unsaturated or aromatic and wherein said carbocycle contains 0, 1 or 2 nitrogen atoms as ring members;
L¹ is selected from the group consisting of C₁₋₆-alkylen; N(H)—S(═O)₂ and S(═O)₂—N(H);
L² is selected from the group consisting of S; O; C₁₋₆ alkylen-C(═O); S—C₁₋₆ alkylen-C(═O); O—C₁₋₆ alkylen-C(═O); C(═O)—C₁₋₆-alkylen; C(═O)—C₁₋₆-alkylen-S; C(═O)—C₁₋₆-alkylen-O; C₁₋₆-alkylen-C(═O)—N(H); S—C₁₋₆-alkylen-C(═O)—N(H); O—C₁₋₆-alkylen-C(═O)—N(H); C(═O)—N(H)—C₁₋₆-alkylen; C(═O)—N(H)—C₁₋₆-alkylen-S; C(═O)—N(H)—C₁₋₆-alkylen-O; C₁₋₆-alkylen-N(H)—C(═O); S—C₁₋₆-alkylen-N(H)—C(═O); O—C₁₋₆-alkylen-N(H)—C(═O); N(H)—C(═O)—C₁₋₆ alkylen; N(H)—C(═O)—C₁₋₆ alkylen-S; N(H)—C(═O)—C₁₋₆ alkylen-O; C₁₋₆-alkylen-N(H)—S(═O)₂; S—C₁₋₆-alkylen-N(H)—S(═O)₂; O—C₁₋₆-alkylen-N(H)—S(═O)₂; N(H)—S(═O)₂—C₁₋₆-alkylen; N(H)—S(═O)₂—C₁₋₆-alkylen-S; N(H)—S(═O)₂—C₁₋₆-alkylen-O; C₁₋₆-alkylen-S(═O)₂—N(H); S—C₁₋₆-alkylen-S(═O)₂—N(H); O—C₁₋₆-alkylen-S(═O)₂—N(H); S(═O)₂—N(H)—C₁₋₆-alkylen; S(═O)₂—N(H)—C₁₋₆-alkylen-S; S(═O)₂—N(H)—C₁₋₆-alkylen-O;
   with the proviso that L¹ and L² are linked to vicinal ring-members of the carbocycle;
R¹, R² are each selected from the group consisting of —H; halogen; C₁₋₆ alkyl; C₂₋₆ alkenyl; C₂₋₆ alkynyl; —O—C₁₋₆ alkyl; —S—C₁₋₆ alkyl; C₁₋₆-haloalkyl; —O—C₁₋₆ haloalkyl; —S—C₁₋₆-haloalkyl; —OH; —SH; —CN; —NO₂ and —NR^{a}R^{b}, wherein R^{a} and R^{b} are independently H or C₁₋₆ alkyl;
   with the proviso that at least one of R¹ and R² represents H;
R³, R⁴ are each selected from the group consisting of —H; halogen; C₁₋₆ alkyl; C₂₋₆ alkenyl; C₂₋₆ alkynyl; —O—C₁₋₆ alkyl; —S—C₁₋₆ alkyl; C₁₋₆-haloalkyl; —O—C₁₋₆ haloalkyl; —S—C₁₋₆-haloalkyl; —OH; —SH; —CN; —NO₂ and —NR^{a}R^{b}, wherein R^{a} and R^{b} are independently H or C₁₋₆ alkyl;
   with the proviso that at least one of R³ and R⁴ does not represent H;
R⁵, R⁶ mutually independent, are each selected from the group consisting of —H; ═O; halogen; C₁₋₆ alkyl; C₂₋₆ alkenyl; C₂₋₆ alkynyl; —O—C₁₋₆ alkyl; —S—C₁₋₆ alkyl; C₁₋₆-haloalkyl; —O—C₁₋₆ haloalkyl; —S—C₁₋₆-haloalkyl; —OH; —SH; —CN; —NO₂ and —NR^{a}R^{b}, wherein R^{a} and R^{b} are independently H or C₁₋₆ alkyl.

In a preferred embodiment, the present invention relates to a pharmaceutical composition comprising one or more compounds of general formula IA or a pharmaceutically acceptable salt thereof:

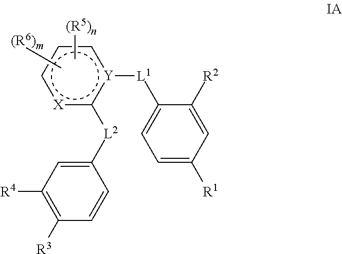

wherein
X, Y mutually independent, each represent a carbon atom or a nitrogen atom;

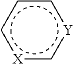

represents a 6-membered carbocycle, wherein said carbocycle is partially unsaturated or aromatic,
   and
m, n, L¹, L², R¹, R², R³, R⁴, R⁵, R⁶ have the meaning as described above.

It is preferred that X and Y do not both represent a carbon atom.

In another preferred embodiment, the present invention relates to a pharmaceutical composition comprising one or more compounds of general formula IB or a pharmaceutically acceptable salt thereof:

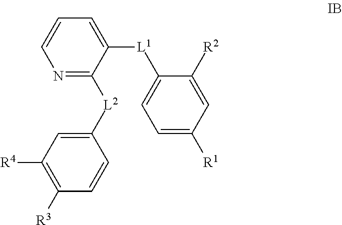

wherein L¹, L², R¹, R², R³ and R⁴ have the meaning as described above.

In a particularly preferred embodiment, the present invention relates to a pharmaceutical composition comprising one or more compounds of general formula IC or a pharmaceutically acceptable salt thereof:

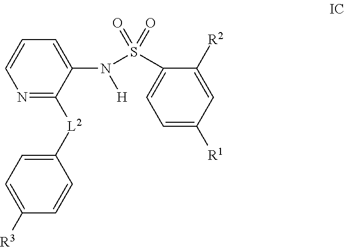

wherein
L² represents S or O;
R¹, R² are each selected from the group consisting of —H; —F; —Cl; —Br; methyl; ethyl; n-propyl; iso-propyl; n-butyl; sec-butyl; iso-butyl; tert-butyl; methoxy; ethoxy; —CF₃; —OCF₃; —SCF₃; —OH and —CN;
   with the proviso that at least one of R¹ and R² represents H, and
R³ represents —F, —Cl, —Br; methyl, ethyl, n-propyl, iso-propyl, n-butyl, sec-butyl, iso-butyl, tert-butyl; methoxy, ethoxy, —CF₃, —OCF₃, —SCF₃; —OH and —CN.

In the afore mentioned compounds of formula IC it may be preferred that one of R¹ and R² represents H while the other of R¹ and R² is selected from the list of possible substituents.

In another preferred embodiment, the present invention relates to a pharmaceutical composition comprising one or more compounds of general formula ID or a pharmaceutically acceptable salt thereof:

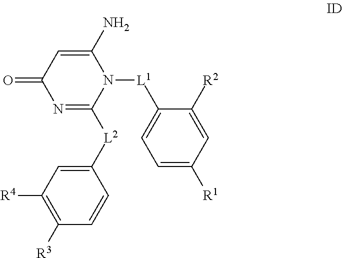

wherein L¹, L², R¹, R², R³ and R⁴ have the same meaning as described above.

In yet another particularly preferred embodiment, the present invention relates to a pharmaceutical composition comprising one or more compounds of general formula IE or a pharmaceutically acceptable salt thereof:

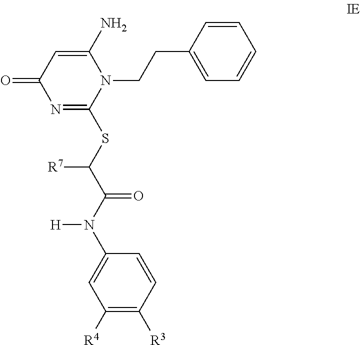

wherein
R⁷ is selected from the group consisting of H; methyl; ethyl; n-propyl; iso-propyl; n-butyl; iso-butyl; sec-butyl and tert-butyl;
R³, R⁴ are each selected from the group consisting of —H; —F, —Cl, —Br; methyl; ethyl; n-propyl; iso-propyl; n-butyl; sec-butyl; iso-butyl; tert-butyl; methoxy; ethoxy; —CF₃; —OCF₃; —SCF₃; —OH and —CN;
   with the proviso that one of R³ and R⁴ represents H while the other of R³ and R⁴ does not represent H.

In any of the compounds of formulae I, IA, IB, IC and ID it may be preferred that the substituent(s) on one phenyl ring (e.g. R¹, R²) may give rise to a polar character of said ring, while the substituents on the other phenyl ring (e.g. R³, R⁴) may give rise to an unpolar character of the ring, or vice versa. Suitable substituents for inducing a polar character are, for example, halogen; —O—C₁₋₆ alkyl; —S—C₁₋₆ alkyl; C₁₋₆-haloalkyl; —O—C₁₋₆ haloalkyl; —S—C₁₋₆-haloalkyl; —OH; —SH; —CN; —NO₂ and —NR^{a}R^{b}, wherein R^{a} and R^{b} are independently H or C₁₋₆ alkyl. In particular, such substituents are halogen such as —F and —Cl and C₁₋₆-haloalkyl such as —CF₃. Suitable substituents for inducing an unpolar character to the ring are for example C₁₋₆ alkyl; C₂₋₆ alkenyl and C₂₋₆ alkynyl.

In a most preferred embodiment, the present invention relates to a pharmaceutical composition comprising one or more compounds selected from the group consisting of:
[1] N-(2-(4-chlorophenoxy)pyridin-3-yl)-4-isopropylbenzenesulfonamide,
[2] N-(2-(4-tert-butylphenoxy)pyridin-3-yl)-2-(trifluoromethyl)benzenesulfonamide,
[3] 4-chloro-N-(2-(p-tolylthio)pyridin-3-yl)benzenesulfonamide,
[4] 2-(6-amino-4-oxo-1-phenethyl-1,4-dihydropyrimidin-2-ylthio)-N-(3-chlorophenyl)propanamide and
[5] 2-(6-amino-4-oxo-1-phenethyl-1,4-dihydropyrimidin-2-ylthio)-N-(4-chlorophenyl)acetamide.

Where the compounds used according to the present invention have at least one asymmetric center (e.g. compounds of formula IE with R⁷ being different from hydrogen) they may exist as enantiomers. Where the compounds have two or more asymmetric centers, they may additionally exist as diastereoisomers. All such isomers and mixtures thereof in any proportion are also encompassed within the scope of the present invention. Methods for obtaining such stereoisomers and their mixtures are well known to those skilled in the art. The compounds used according to the present invention may also form solvates such as hydrates, which are also within the scope of the present invention.

Those skilled in the art understand that some of the inventively used compounds will be acidic in nature, e.g. those compounds that possess a phenolic hydroxyl group. These compounds may form pharmaceutically acceptable salts. Examples of such salts may include sodium, potassium, calcium salts or salts with amines such as alkyl amines. Certain basic compounds also form pharmaceutically acceptable salts, e.g. acid addition salts. For example, the pyrido-nitrogen atoms may form salts with strong acid, while compounds having basic substituents such as amino groups also form salts with weaker acids. Examples of suitable acids for salt formation include but are not limited to hydrochloride acid, sulfuric acid, phosphoric acid, acetic acid, citric acid, oxalic acid, malonic acid, salicylic acid, malic acid, fumaric acid, succinic acid, ascorbic acid, maleic acid, methanesulfonic acid and other mineral and carboxylic acids well known to those skilled in the art. Methods for obtaining salts are also well known to those skilled in the art.

The pharmaceutical compositions may also comprise one or more additional active agents that may be useful when treating a certain disease. For example, when treating cancer, in particular multiple myeloma, other chemotherapy drugs may be used in combination with the inventively used gamma secretase inhibitors. Examples of such chemotherapy drugs include alkylating agents such as melphalan or proteasome inhibitors such as bortezomib.

The compounds of formulae I, IA, IB, IC, ID and IE are either commercially available, e.g. from Maybridge, Acros Organics, Geel, Belgium or Ambinter SARL, Paris, France or may be prepared by methods well known to those skilled in the art, e.g. as disclosed in US 2007/0037794; El-Sabbagh et al. Bollettino Chimico Farmaceutico 1995, 134, 80-84 and WO 2005/037779, or analogues methods.

The inventive pharmaceutical compositions are particularly useful for the treatment of renal disorders, wherein said renal disorders may preferably be selected from the group consisting of kidney failure; podocyte damage; glomerular diseases, in particular focal glomerulosclerosis or segmental glomerulosclerosis, and diabetic nephropathy.

Moreover, the inventive pharmaceutical compositions are particularly useful for the treatment of cancer, wherein said cancer may preferably be selected from the group consisting of renal cancer, multiple myeloma, leukemia and colon cancer.

Furthermore, the pharmaceutical compositions according to the present invention are also useful for treatment of neurodegenerative disorders, wherein said neurodegenerative disorders may preferably be selected from the group consisting of Morbus Alzheimer, disorders associated with the deposition of beta-amyloid, age-related dementia, cerebral amyloidosis, systemic amyloidosis, hereditary cerebral hemorrhage with amyloidosis, Down's syndrome and ischemic stroke.

A further aspect of the present invention relates to the use of one or more of the compounds described herein for the manufacture of a medicament.

In yet another one of its aspects the present invention relates to the use of one or more of the compounds described herein for the manufacture of a medicament for the treatment of renal disorders, wherein said renal disorders may preferably be selected from the group consisting of kidney failure; podocyte damage; glomerular diseases, in particular focal glomerulosclerosis or segmental glomerulosclerosis, and diabetic nephropathy.

In yet another one of its aspects the present invention relates to the use of one or more of the compounds described herein for the manufacture of a medicament for the treatment of cancer, wherein said cancer may preferably be selected from the group consisting of renal cancer, multiple myeloma, leukemia and colon cancer.

In yet a further aspect the present invention relates to the use of one or more of the compounds described herein for the manufacture of a medicament for the treatment of neurodegenerative disorders, wherein said neurodegenerative disorders may preferably be selected from the group consisting of Morbus Alzheimer, disorders associated with the deposition of beta-amyloid, age-related dementia, cerebral amyloidosis, systemic amyloidosis, hereditary cerebral hemorrhage with amyloidosis, Down's syndrome and ischemic stroke.

Yet another aspect of the present invention relates to a method of modulating, e.g. inhibiting, gamma secretase in a patient in need of such treatment comprising administering to said patient an effective amount of one or more compounds as described herein.

Yet another aspect of the present invention relates to a method of inhibiting the deposition of beta amyloid protein in a patient in need of such treatment comprising administering to said patient an effective amount of one or more compounds as described herein.

A further aspect of the present invention relates to a method of treating renal disorders in a patient in need of such treatment comprising administering to said patient an effective amount of one or more compounds as described herein. The renal disorders may preferably be selected from the group given above.

Another aspect of the present invention relates to a method of treating cancer in a patient in need of such treatment comprising administering to said patient an effective amount of one or more compounds as described herein. The cancer may preferably be selected from the group given above.

Yet another aspect of the present invention relates to a method of treating neurodegenerative disorders in a patient in need of such treatment comprising administering to said patient an effective amount of one or more compounds as described herein. The neurdegenerative disorders may preferably be selected from the group given above.

The term patient as used herein includes humans as well as mammals.

The notch signaling pathway and gamma secretase play a role in many organs and tissues, for example, in the eye, kidney, pancreas, prostate, mammae, liver, gall bladder, and mucosa. The inventive pharmaceutical compositions may be formulated to specifically target certain tissues and/or organs.

It is to be understood that the term pharmaceutical composition as used herein includes one or more of the compounds as described herein as medicament. Said term further encompasses mixtures of one or more of the compounds as described herein with one or more additional active agents and/or one or more pharmaceutically acceptable carriers.

The pharmaceutical composition (medicament) according to the present invention may in addition to one or more of the compounds described herein comprise one or more pharmaceutically acceptable carriers. Such pharmaceutically acceptable carriers can be either solid, semi-solid or liquid.

The inventive pharmaceutical compositions may be applied via topical/local or parenteral administration.

The inventive pharmaceutical preparations may preferably be formulated for parenteral administration, thereby including intravenous, intraarterial, intramuscular, subcutaneous, intradermal, intrathecal, intraperitoneal, transdermal, transmucosal (sublingual, buccal) and inhalational administration. Parenteral administration includes administration via injection as well as infusion.

Solid form preparations include powders; multiparticulates such as pellets, granules, or crystals; tablets, pills, capsules, cachets and suppositories.

The powders, multiparticulates, pills and tablets may be comprised of from about 1 to about 99, preferably 5 to about 95, percent active compound. Suitable solid carriers are known in the art, e.g. magnesium carbonate, magnesium stearate, talc, sugar or lactose. Tablets, pills powders, multiparticulates, cachets and capsules can be used as solid dosage forms suitable for oral administration. Oral dosage forms may also release the active substance(s) in a delayed manner.

The inventive pharmaceutical compositions may also be in form of a liposomal preparation, preferably for oral or parenteral administration. The inventive pharmaceutical compositions may also be in a form of an organ and/or tissue targeted preparation, preferably for oral or parenteral administration. For example, said preparation may be an organ targeted liposomal preparation for parenteral administration.

Examples of pharmaceutically acceptable carriers and methods of manufacture for various compositions may be found in A. Gennaro (ed.), Remington's Pharmaceutical Sciences, 18th Edition, (1990), Mack Publishing Co., Easton, Pa.

Liquid form preparations include solutions, suspensions and emulsions. As an example may be mentioned water or water-propylene glycol solutions for parenteral injection or addition of sweeteners and opacifiers for oral solutions, suspensions and emulsions. Liquid form preparations may also include solutions for intranasal administration.

Aerosol preparations suitable for inhalation may include solutions and solids in powder form, which may be in combination with a pharmaceutically acceptable carrier, such as an inert compressed gas, e.g. nitrogen.

Also included are solid form preparations which are intended to be converted, shortly before use, to liquid form preparations for either oral or parenteral administration.

Such liquid forms include solutions, suspensions and emulsions.

The compositions of the invention may also be deliverable transdermally. The transdermal compositions can, for example, take the form of creams, lotions, aerosols and/or emulsions and can be included in a transdermal patch of the matrix or reservoir type as are conventional in the art for this purpose.

The compositions of the invention may also be deliverable subcutaneously.

Preferably, the inventive pharmaceutical composition is a medicament e.g. in a unit dosage form. In such form, the composition is subdivided into suitably sized unit doses containing appropriate quantities of the active compound, e.g., an effective amount to achieve the desired purpose.

The quantity of active compound in a unit dose may be varied or adjusted from about 0.01 mg to about 1000 mg, preferably from about 0.01 mg to about 750 mg, more preferably from about 0.01 mg to about 500 mg, according to the particular application.

The actual dosage employed may be varied depending upon the requirements of the patient and the severity of the condition being treated. Determination of the proper dosage regimen for a particular situation is within the skill of the art. For convenience, the total daily dosage may be divided and administered in portions during the day as required.

The amount and frequency of administration of the compounds used according to the invention and/or the pharmaceutically acceptable salts thereof will be regulated according to the judgment of the attending clinician considering such factors as age, condition and size of the patient as well as severity of the symptoms being treated. A typical recommended daily dosage regimen for oral administration can range from about 0.04 mg/day to about 4000 mg/day, in one or more, e.g. one to four divided doses.

### Pharmacological Methods

### Cell Culture

Human MM cell lines used were as follows: NCI-H929, OPM-2, LP-1, RPMI-8226, U266 (DSMZ, Braunschweig, Germany). Cell lines were cultured in RPMI 1640 medium (Biochrom, Berlin, Germany) supplemented with 10% heat-inactivated fetal calf serum (FCS, Gibco, Karlsruhe, Germany), 1 mM sodium pyruvate and 100 units/ml penicillin and 100 μg/ml streptomycin (Gibco). Human osteoclasts were obtained as described in the reference of Zavrski I, Krebbel H, Wildemann B, et al. Proteasome inhibitors abrogate osteoclast differentiation and osteoclast function. Biochem Biophys Res Commun. 2005; 333:200-205. Briefly, peripheral blood mononuclear cells (PBMC) were isolated from whole blood of healthy volunteers, using a Ficoll-Hypaque density gradient. The adherent fraction of the isolated mononuclear cells was cultured for 21 days in MEM medium (Sigma-Aldrich Chemie, Taufkirchen, Germany) supplemented with 10% FCS, 100 U/ml penicillin, 100 μg/ml streptomycin, 50 ng/ml M-CSF, and 25 ng/ml RANKL (osteoclastogenic medium), before coculture experiments and treatment with the inventive compounds started. Human osteoblasts were obtained according to Robey P G, Termine J D. Human bone cells in vitro. Calcif Tissue Int. 1985; 37:453-460 as described in Hecht M, Heider U, Kaiser M, von Metzler I, Sterz J, Sezer O. Osteoblasts promote migration and invasion of myeloma cells through upregulation of matrix metalloproteinases, urokinase plasminogen activator, hepatocyte growth factor and activation of p38 MAPK. Br J Haematol. 2007; 138: 446-458. Briefly, trabecular bone specimens from patients without malignant disease undergoing knee or hip surgery were minced and washed to remove bone marrow cells. The bone fragments were resuspended in Dulbecco's modified Eagle's medium (DMEM)/HAM's F12 medium (Biochrom, Berlin, Germany) supplemented with 10% FCS and cultured in tissue culture flasks until a confluent cell monolayer was obtained. Culturing of functional osteoblasts was confirmed by alkaline phosphatase (ALP) staining (kit from Sigma, USA), realtime RT-PCR analysis of expression of osteoblast markers (ALP, osteocalcin) and by von-Kossa staining before coculture and treatment experiments were started.

### Drug Treatment of MM Cells

The compound according to example 1, hereinafter referred to as GSI15, (compound RH02015SC, Maybridge, Acros Organics, Geel, Belgium) was freshly dissolved as 26 mM stock solution in Dimethylsulfoxide (DMSO). Multiple myeloma therapeutics used were melphalan (marketed as Alkeran®, GlaxoSmithKline, Munich, Germany) and bortezomib (marketed as Velcade®, Janssen-Cilag, Neuss, Germany). Melphalan was freshly dissolved at 10 mg/ml in 0.9% NaCl-solution. Bortezomib was freshly dissolved at 100 ng/ml in 0.9% NaCl solution. Gamma Secretase Inhibitor (GSI) treatment for apoptosis assessment by AnnexinV/PI staining, cell cycle analysis and coculture experiments was performed in 6-well plates. GSI15 stock solution was added directly to the cell suspension. GSI treatment for assaying proliferation was performed in 96-well plates. 10,000 cells per well were seeded in 50 μl medium per well before 50 μl of a 2× solution of GSI15 prediluted in medium were added per well. Melphalan and bortezomib treatment as well as their combination with GSI15 for assaying apoptosis by AnnexinV/PI staining was done in 6-well plates. All inhibitors were added directly to the cells in 6-well plates.

### Coculture Experiments

In osteoclast coculture experiments, OPM-2 cells (1×10⁶ cells) were added to osteoclasts in 60 mm dishes (4×10⁶ cells per dish) in 3 ml MEM medium supplemented with 10% FCS, 100 U/ml penicillin, 100 μg/ml streptomycin. GSI15 (30 μM, 60 μM) or DMSO (equivalent to 60 μM) as solvent control were added to each well (daily treatment). After 48 h, 0.5 ml OPM-2 cell suspension were harvested per well and subjected to AnnexinV-FITC/PI staining. Remaining OPM-2 cells were harvested and lysed for either RNA or protein preparation. The monolayer of osteoclasts was washed twice with ice-cold PBS and then lysed for either RNA or protein preparation directly on the plate.

In osteoblast coculture experiments, OPM-2 cells (7.5×10⁵) were added to osteoblasts (2×10⁵ cells) in 6-well plates in 2 ml DMEM/HAM's F12 medium supplemented with 10% FCS, 100 U/ml penicillin, 100 μg/ml streptomycin. GSI15 (40 μM, 60 μM, 80 μM) or DMSO (equivalent to 80 μM) as solvent control was added to each well (daily treatment). After 72 h of coculture and GSI15 treatment OPM-2 cells in suspension were aspirated and subjected to protein lysis. The remaining monolayer of osteoblasts was washed twice with ice-cold PBS and lysed directly on the plate with protein lysis buffer.

### Immunoblotting

Whole cell extracts were prepared and quantified as described in Jundt F, Anagnostopoulos I, Forster R, Mathas S, Stein H, Dorken B. Activated Notch1 signaling promotes tumor cell proliferation and survival in Hodgkin and anaplastic large cell lymphoma. Blood. 2002; 99:3398-3403. Proteins (30 μg) were resolved by sodium dodecyl sulfate-polyacrylamide gel electrophoresis (SDS-PAGE) and transferred to nitrocellulose membranes. Protein loading was normalized by Ponceau red staining. Membranes were incubated with monoclonal mouse anti-Notch1 antibody (no. 552466; BD Biosciences, Heidelberg, Germany), anti-Jagged1 antibody (sc-8303; Santa Cruz Biotechnology), polyclonal rabbit anti-Delta antiboday (Santa Cruz Biotechnology), polyclonal rabbit anti-cleaved PARP (Asp214) antibody (Cell Signaling Technologies, Frankfurt a.M., Germany) or mouse monoclonal anti-tubulin antibody (Sigma, Deisenhofen, Germany).

Secondary antibodies were goat anti-mouse (Promega, Mannheim, Germany), goat anti-rat (Dianova, Hamburg, Germany), and goat anti-rabbit (Santa Cruz Biotechnology) horseradish peroxidase (HRP)-conjugated antibodies. Detection was done using Pico chemi-luminescence reagent (Perbio Science, Bonn, Germany).

### Quantitative RT-PCR Analysis

Quantitative real-time reverse transcription-PCR (RT-PCR) analysis was performed using the following primer/probe sets: human Hes-1 (forward—CCCGTCTACCTCTCTCCTTG, reverse—GAGCAAGTGCTGAGGGTTTA, probe—FAM-CCTGGAACAGCGCTACTGATCACC-TAMRA) human TRAP5 (forward—AGATCCTGGGTGCAGACTTC, reverse—AAGGGAGCGGTCAGAGAATA, probe FAM-CGTCCTCAAAGGTCTCCTGGAACC-TAMRA), human beta2-microglobulin (forward—ccc cca ctg aaa aag atg ag, reverse—atc caa tcc aaa tgc ggc, probe—FAM-CCT GCC GTG TGA ACC ATG TGA CTT T-TAMRA) served as normalizer. Total RNA was extracted from MM cells, human osteoclasts or human osteoblasts using RNeasy Mini Kit (Qiagen, Hilden, Germany) following the manufacturer's instructions. For the RT-PCR reaction SuperScript™ III Platinum® One-Step Quantitative RT-PCR System (Invitrogen, Karlsruhe, Germany) was used. 50 ng total RNA were used per reaction, each RNA sample was analyzed in three replicates. PCR conditions (40 cycles) were the following: reverse transcription at 50° C. for 30 minutes, initial denaturation at 95° C. for 10 minutes, followed by 40 cycles of 45 seconds denaturation at 95° C. and 60 seconds annealing/extension at 62° C. Amplification of the house-keeping gene beta2-microglobulin was used to normalize the expression data. Normalized mRNA expression data were then calculated as values relative to the respective untreated sample.

### Proliferation Assay

For assaying proliferation/viability of cells CellTiter-Glo Luminescent Cell Viability Assay (Promega, Mannheim, Germany) was used. In this assay the number of viable cells at a given time point is determined by quantification of ATP present, which serves as a measure of metabolically active cells. The assay was carried out according to manufacturer's protocol. Briefly, cells were plated in 96-well plates at 10,000 cells per well in 100 μl medium and treated with indicated amounts of GSI15, with DMSO as solvent control or left untreated. Each treatment was done in four independent replicates in different wells. 24 h and 48 h after start of treatment 30 μl per well were transferred into an opaque-walled plate and lysed using CellTiter-Glo solution. Luminescence was recorded and integrated for two seconds per well. Average values were calculated and normalized to the respective untreated sample.

### Cell Cycle Analysis and Assessment of Apoptosis

For cell cycle analysis 2×10⁵ cells were spun down, medium was aspirated and cells washed with PBS. Cells were then resuspended in 250 μl PBS. 1 ml ice-cold 70% ethanol was added, followed by overnight incubation at −20° C. Cells were spun down, the pellet washed with PBS and then resuspended in 200 μl PBS containing 0.15 mg/ml RNaseA and 30 μg/ml propidium iodide (PI). After 10 minutes incubation in the dark at room temperature, cells were analyzed by flow cytometry. The amount of apoptotic cells was determined by AnnexinV/propidium iodide staining using Human AnnexinV-FITC Kit (Bender Medsystems, Vienna, Austria), according to manufacturer's protocol. Briefly, 2×10⁵ cells were spun down and washed with PBS, followed by 10 minutes incubation in binding buffer containing AnnexinV-FITC conjugate. Cells were then spun down again, resuspended in binding buffer containing PI and analyzed by flow cytometry.

Cell Culture Experiments with Human Podocyte Cell Line

An imortalized human podocyte cell line (Saleem et al., “A conditionally immortalized human podocyte cell line demonstrating nephrin and podocin expression”, J. Am. Soc. Nephrol. 13: 630-638, 2002) was stimulated with 5 ng/ml TGFβ in the presence or absence of the indicated concentrations of gamma secretase inhibitors. The percentage of the apoptotic cells was determined after 24 hours using the Cell Death Detection ELISA (Roche Diagnostics, Mannheim, Germany) according to the manufaturer's protocol.

### In Vivo Studies (PAN-Model)

For the in vivo studies male Sprague-Dawley rats were purchased from Charles River and treated with puromycin aminoglycoside (PAN) to induce albuminuric glomerular disease as described previously (Niranjan et al., “The Notch pathway in podocytes plays a role in the development of glomerular disease.” Nat. Med. 14: 290-298, 2008). Rats were injected PAN (150 mg/kg) once. The test compounds were administered intraperitoneally (500 μg/100 g rat) once per day (n=4 animals per group). Injection of test compounds were started one day after PAN injection. All studies were performed in accordance with the European Union normative. As a positive control one group of rats received gamma secretase inhibitor DAPT.

Urinary albumine was measured at the indicated days as described previously (Sanchez-Nino et al., “The MIF receptor CD74 in diabetic podocyte injury”. J. Am. Soc. Nephrol. 20: 353-362, 2009). For total protein measurement urine samples were centrifuged and pellets were diluted in distilled water. Exton reagent (230 mM sulfosalic acid, 1.4 M sodium sulfate) was added and after 10 minutes absorbance was measured at 620 nm.

### EXAMPLES

The following example compounds were obtained and tested for their activity as gamma secretase inhibitors.

### Example 1

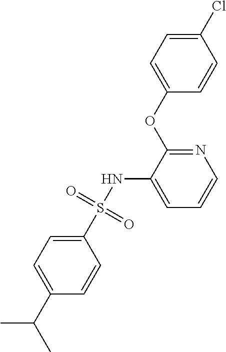

The compound N-(2-(4-chlorophenoxy)pyridin-3-yl)-4-isopropylbenzenesulfonamide was obtained from Maybridge, Acros Organics, Geel, Belgium (No. RH 02015). Said compound may also be obtained by methods as described in US 2007/0037794.

### Example 2

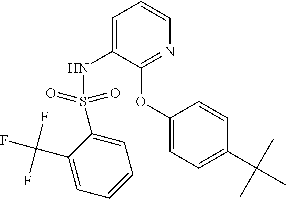

The compound N-(2-(4-tert-butylphenoxy)pyridin-3-yl)-2-(trifluoromethyl)benzenesulfonamide was obtained from Maybridge, Acros Organics, Geel, Belgium (No. RH 02081). Said compound may also be obtained by methods as described in US 2007/0037794.

### Example 3

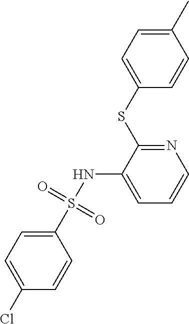

The compound 4-chloro-N-(2-(p-tolylthio)pyridin-3-yl)benzenesulfonamide was obtained from Maybridge, Acros Organics, Geel, Belgium (No. RH 02105). Said compound may also be obtained by methods as described in US 2007/0037794 and El-Sabbagh et al. Bollettino Chimico Farmaceutico 1995, 134, 80-84.

### Example 4

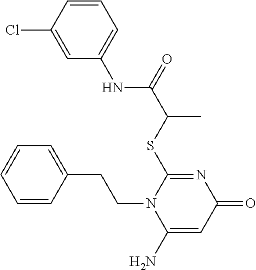

The compound 2-(6-amino-4-oxo-1-phenethyl-1,4-dihydropyrimidin-2-ylthio)-N-(3-chlorophenyl)propanamide was obtained from Ambinter SARL, Paris, France (No. A3593/0152314). Said compound may also be obtained by methods as described in WO 2005/037779.

### Example 5

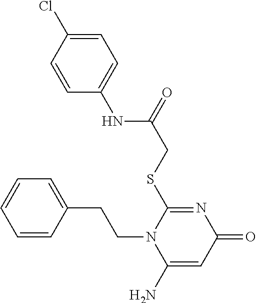

The compound 2-(6-amino-4-oxo-1-phenethyl-1,4-dihydropyrimidin-2-ylthio)-N-(4-chlorophenyl)acetamide was obtained from Ambinter SARL, Paris, France (No. A3144/0132920). Said compound may also be obtained by methods as described in WO 2005/037779.

### Pharmacological Results:

### The Gamma-Secretase Inhibitor (GSI15) Blocks Notch Signaling and Inhibits Proliferation of MM Cells

Analysis of protein expression of Notch1 and the two Notch ligands Jagged1 and Delta in five MM cell lines showed that Notch1 was expressed in four of five MM cell lines, but not in U266 cells as was described in Jundt F, Probsting K S, Anagnostopoulos I, et al. Jagged1-induced Notch signaling drives proliferation of multiple myeloma cells. Blood. 2004; 103:3511-3515. Jagged1 was highly expressed in three of the five cell lines, weakly expressed in RPMI-8226 cells and not expressed in U266 cells. Another Notch1 ligand, Delta, was expressed in all cell lines tested. It has been shown earlier that MM cell proliferation increases when Notch signaling is activated through heterotypic interactions with Jagged1-expressing Hela cells [cf. Jundt F et al., Blood. 2004; 103:3511-3515]. Expression of both Notch and its ligands in four MM cell lines hinted to a potential constitutive activation of the Notch pathway due to activation by homotypic MM cell interactions. In order to evaluate the impact of gamma-secretase inhibition on Notch signaling in MM cells, OPM-2 cells were treated with GSI15. Analysis of Notch1 target gene expression revealed expression of Hes-1 that was dose-dependently downregulated upon GSI15 treatment. This finding supports the notion that there is Notch activity in MM cells that can be specifically downregulated by GSI15. To analyze the tumor biologic effect of GSI15 OPM-2 and LP-1 cells were treated with different doses of GSI15. The U266 cell line lacking Notch1 expression served as a negative control. Notch1 positive OPM-2 and LP-1 cells showed a dose-dependent reduction of proliferation after 48 h treatment-, with the maximum effect at 60 μM GSI15. In contrast, U266 cells were almost not affected by this treatment. Resistance of U266 cells to GSI15 treatment is a further hint that the growth inhibitory effect of the treatment in OPM-2 and LP-1 cells is not due to general toxicity of the compound but rests upon specific Notch inhibition. These results confirm the Notch1-dependent growth behavior of MM cells as suggested by earlier findings [cf. Jundt F et al., Blood. 2004; 103:3511-3515]. GSI15 can be used to inhibit MM cell growth.

The concentration at which 50% growth inhibition was observed was determined for example compounds 1-5 in the assay described above. The results are given in the following table:

| | Concentration at which 50% cell |
|---|---|
| | growth inhibition was observed |
| Example compound | [micro M] |
| 1 | 20 |
| 2 | 30 |
| 3 | 30 |
| 4 | 20 |
| 5 | 20 |

### Gamma-Secretase Inhibition Induces Apoptosis in MM Cell Lines

Furthermore, it was analyzed whether the growth inhibitory effect of GSI15 is due to a cell-cycle arrest or apoptosis induction. To that end, MM cells were treated with GSI15 and subjected to propidium iodide staining after different incubation times. Interestingly, the distribution of G1-, S- and G2-phase remained unaltered after 48 h and 72 h, while GSI15 treatment resulted in an increase in the sub-G1 fraction in OPM-2 cells and LP-1 cells after 72 h, indicating induction of apoptosis. In U266 cells no change was detected. Data was confirmed by analyzing GSI15 treated MM cells by AnnexinV-FITC/PI staining. Analyzed were Notch1 expressing OPM-2 cells and the U266 cell line lacking Notch1 expression, that would be expected to be insensitive to inhibition of Notch signaling and served thus as a control. 48 h of treatment resulted in a decrease of OPM-2 cells negative for both Annexin V-FITC- and PI-staining. Instead both early apoptotic cells (Annexin V-FITC-positive) and late apoptotic cells (Annexin V-FITC- and PI-positive) appeared in the culture. There was no change in AnnexinV-positive but PI-negative cells that would resemble necrosis rather than apoptosis, thus unspecific toxicity of the inhibitor is less probable. As expected, U266 cells remained unaffected by Gamma secretase inhibition. Furthermore, protein lysates of GSI15 treated MM cells were examined by Western blotting for the emergence of a poly-(ADP-ribose) polymerase (PARP) cleavage product that is commonly used as a marker of apoptosis. Apoptosis induction could be shown in Notch1 expressing OPM-2 cells already with 30 μM GSI15 but not in the U266 cell line. Thus, evidence is provided that GSI15 specifically acts on the Notch pathway and exerts its growth reducing effect on MM cell lines via induction of apoptosis.

GSI15 Synergizes with Bortezomib and Melphalan Inducing Apoptosis in MM Cells

The alkylating agent melphalan and the proteasome inhibitor bortezomib are in clinical use to treat MM patients either alone or in combination [cf. Ghobrial I M, Leleu X, Hatjiharissi E, et al. Emerging drugs in multiple myeloma. Expert Opin Emerg Drugs. 2007; 12:155-163.]. While bortezomib has been used with great success in patients previously refractory to treatment, increasing their progression-free and overall survival rates, there is still no curative therapeutic approach. Therefore, the development of novel drugs might be a necessary step towards more successful therapy. The effect of GSI15 treatment on MM cell growth in combination with bortezomib and melphalan was investigated. For this purpose, OPM-2 cells were cultured with low doses of either bortezomib or melphalan and GSI15. Apoptosis induction in the OPM-2 cells was measured by AnnexinV-FITC/PI staining. The numbers of viable cells after 24 h or 48 h treatment normalized to vehicle treated control cells were determined. As expected, both melphalan and bortezomib dose-dependently triggered cell death. GSI15 alone at the low dose of 40 μM did not induce apoptosis after 24 h and merely slightly induced apoptosis in OPM-2 cells after 48 h (24% apoptotic cells). However, GSI15 doubled melphalan-induced apoptosis after 24 h (32% apoptotic cells by 50 μM melphalan alone compared to 63% apoptotic cells by the combination). After 48 h treatment, both 40 μM GSI15 and 2 nM bortezomib alone led to −25% apoptotic cells, whereas their combination yielded 75% apoptotic cells. Thus, GSI15 dramatically augmented the effect of bortezomib or melphalan and synergistically induced apoptosis in OPM-2 cells.

Activation of Notch Signaling and Increased Activity of OCL after Coculture with MM Cells

MM cells were cocultured with either human OCL or OBL and analyzed Notch signaling in order to evaluate its impact on tumor-stroma interactions. Whereas OCL and OPM-2 cells expressed Notch1 protein, only the OPM-2 cells also expressed the Notch1 ligands Jagged1 and Delta. This finding is in line with the observation that OPM-2 cells alone exhibit expression of the Notch target gene Hes-1 possibly due to homotypic interactions. Analysis of mRNA expression of the Notch target Hes-1 in cocultured cells revealed no change in the OPM-2 cells but a 2.3 fold increase in OCL, suggesting specific activation of Notch signaling in OCL through interaction with MM cells. Next, cocultures with OBL were analyzed, obtained from outgrowth cultures of bone biopsies. Analysis of protein expression in OPM-2 and OBL revealed that the two cell types express both Notch1 and its ligands Jagged1 and Delta. Accordingly, there was no change in pathway activity detectable as measured by Hes-1 mRNA expression, neither in OPM-2 cells nor in OBL. These data suggest that MM cells can upregulate Notch signaling activity in OCL, but not in OBL.

To analyze whether increased Notch activity has functional consequences, resulting in enhanced OCL activity, GSI15 was utilized in the OPM-2/OCL coculture system. After 48 h apoptosis in OPM-2 cells was assayed by AnnexinV-FITC/PI staining. OPM-2 cells alone became apoptotic as shown in the earlier experiments. Interestingly, apoptosis induction through GSI15 was even more pronounced in cocultured OPM-2 cells. At 60 μM GSI15 the overall amount of apoptotic cells increased from 29% in OPM-2 alone to 83% in OPM-2 cocultured with OCL. In addition, apoptosis in both cell types was assessed using cleaved PARP. Strong induction of PARP cleavage occurred in OPM-2 cells in both mono- and coculture, confirming the AnnexinV-FITC/PI staining results. GSI15 treatment did not induce PARP cleavage in OCL alone, whereas it was induced in OCL cocultured with OPM-2 cells at high doses of GSI15. Furthermore, Notch pathway activity in OCL and OCL activity was analyzed by measurement of Hes-1 and tartrate resistant acid phosphatase-5 (TRAP5) expression, respectively. TRAP5 expression correlates with OCL function [cf. Minkin C. Bone acid phosphatase: tartrate-resistant acid phosphatase as a marker of osteoclast function. Calcif Tissue Int. 1982; 34:285-290, Miller S C. The rapid appearance of acid phosphatase activity at the developing ruffled border of parathyroid hormone activated medullary bone osteoclasts. Calcif Tissue Int. 1985; 37: 526-529] and serves as a specific marker of OCL activity. Investigation of mRNA expression revealed upregulation of Hes-1 in OCL upon coculture with OPM-2, confirming our earlier experiments (comparison DMSO treated OCL in mono- and coculture). GSI15 diminished Hes-1 mRNA expression and completely blocked MM cell dependent upregulation of Notch activity in OCL. Interestingly, Hes-1 upregulation in cocultured OCL was accompanied by increased TRAP5 mRNA expression, indicative of higher OCL activity. Intriguingly, GSI15 completely blocked the upregulation of TRAP5 mRNA expression after coculture. This finding points to a contribution of Notch signaling to MM cell-dependent activation of OCL. Our data suggest that GSI15 can induce apoptosis in MM cells and prevent MM cell dependent upregulation of OCL activity.

The consequences of GSI15 treatment on other BMS cells were also analyzed. Cocultures of MM cells and OBL as well as mesenchymal stem cell progenitors of osteoblasts (MSC) were analyzed. Analysis of PARP cleavage by Western blotting revealed apoptosis induction in MM cells in coculture with OBL or MSC. Neither in OBL nor in MSC apoptosis was induced even upon high doses of GSI15 in mono- and cocultures. Accordingly, in a viability assay there was no difference between untreated and GSI15 treated OBL and MSC, whereas the viability of OPM-2 cells was inhibited.

### Physiological Compatibility

The compound according to example 1 was tested for its physiological compatibility via parenteral administration in mice. The LD₅₀ (parenteral, mouse) is 800 mg/kg. Accordingly compound 1 can be considered to be physiologically compatible.

Cell Culture Experiments with Human Podocyte Cell Line

The compounds according to examples 1 and 2 were tested in these cell culture experiments as described above. Both compounds show a positive effect in these experiments.

In cells treated with the gamma secretase inhibitor according to example 1) 41% inhibition of apoptosis was found at 500 nM compared to positive control TGFβ alone. In cells treated with the gamma secretase inhibitor according to example 2) 27% inhibition at 3 μM and 10 μM was found compared to positive control TGFβ alone. In cells treated with the gamma secretase inhibitor DAPT as positive control 37% inhibition at 1 μM was found compared to positive control TGFβ alone.

### PAN-Model

The compounds according to examples 1 and 2 were tested in the PAN-model described above.

It was found that compound 1) and compound 2) show a positive effect on PAN-induced proteinuria and albuminuria on day 7 and day 5, respectively. At day 7 in the group of rats receiving the gamma secretase inhibitor according to example 1) 56% inhibition of albuminuria and 50% inhibition of proteinuria was found compared to positive control PAN alone. At day 5 in the group of rats receiving the gamma secretase inhibitor according to example 2) 42% inhibition of albuminuria and 37% inhibition of proteinuria was found compared to positive control PAN alone.

1. A pharmaceutical composition comprising one or more compounds of formula I or a pharmaceutically acceptable salt thereof: 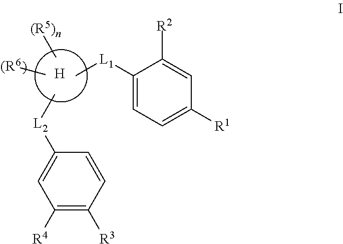
wherein
n, m independently represent 0 or 1; 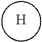
represents a 5, 6 or 7 membered carbocycle, wherein said carbocycle is partially unsaturated or aromatic and wherein said carbocycle contains 0, 1 or 2 nitrogen atoms as ring members;
L¹ is selected from the group consisting of C₂₋₆-alkylene; N(H)—S(═O)₂ and S(═O)₂—N(H);
wherein C₂₋₆-alkylene is selected from the group consisting of —CH₂—CH₂, —CH(CH₃)—, —CH₂—CH₂—CH₂—, —CH(CH₃)—CH₂—, —CH(CH₂CH₃)—, —CH₂—(CH₂)₂—CH₂—, —CH(CH₃)—CH₂—CH₂—, —CH₂—CH(CH₃)—CH₂—, —CH(CH₃)—CH(CH₃)—, —CH(CH₂CH₃)—CH₂—, —C(CH₃)₂—CH₂—, —CH(CH₂CH₂CH₃)—, —C(CH₃)(CH₂CH₃)—, —CH₂—(CH₂)₃—CH₂—, —CH(CH₃)—CH₂—CH₂—CH₂—, —CH₂—CH(CH₃)—CH₂—CH₂, —CH(CH₃)—CH₂—CH(CH₃)—, —CH(CH₃)—CH(CH₃)—CH₂, —C(CH₃)₂—CH₂—CH₂—, —CH₂—C(CH₃)₂—CH₂—, —CH(CH₂CH₃)—CH₂—CH₂—, —CH₂—CH(CH₂CH₃)—CH₂—, —C(CH₃)₂—CH(CH₃)—, —CH(CH₂CH₃)—CH(CH₃), —C(CH₃)(CH₂CH₃)—CH₂—, —CH(CH₂CH₂CH₃)—CH₂—, —C(CH₂CH₂CH₃)—CH₂—, —CH(CH₂CH₂CH₂CH₃)—, —C(CH₃)(CH₂CH₂CH₃)—, —C(CH₂CH₃)₂— and —CH₂—(CH₂)₄—CH₂—.
L² is selected from the group consisting of S; O; C₁₋₆ alkylen-C(═O); S—C₁₋₆ alkylene-C(═O); O—C₁₋₆ alkylene-C(═O); C(═O)—C₁₋₆-alkylene; C(═O)—C₁₋₆-alkylene-S; C(═O)—C₁₋₆-alkylene-0; C₁₋₆-alkylene-C(═O)—N(H); S—C₁₋₆-alkylene-C(═O)—N(H); O—C₁₋₆-alkylene-C(═O)—N(H); C(═O)—N(H)—C₁₋₆-alkylene-S; C(═O)—N(H)—C₁₋₆-alkylene-O; C₁₋₆-alkylene-N(H)—C(═O); S—C₁₋₆-alkylene-N(H)—C(═O); O—C₁₋₆-alkylene-N(H)—C(═O); N(H)—C(═O)—C₁₋₆ alkylene; N(H)—C(═O)—C₁₋₆ alkylene-S; N(H)—C(═O)—C₁₋₆ alkylene-O; C₁₋₆-alkylene-N(H)—S(═O)₂; S—C₁₋₆-alkylene-N(H)—S(═O)₂; O—C₁₋₆-alkylene-N(H)—S(═O)₂; N(H)—S(═O)₂—C₁₋₆-alkylene; N(H)—S(═O)₂—C₁₋₆-alkylene-S; N(H)—S(═O)₂—C₁₋₆-alkylene-O; C₁₋₆-alkylene-S(═O)₂—N(H); S—C₁₋₆-alkylene-S(═O)₂—N(H); O—C₁₋₆-alkylene-S(═O)₂—N(H); S(═O)₂—N(H)—C₁₋₆-alkylene; S(═O)₂—N(H)—C₁₋₆-alkylene-S; S(═O)₂—N(H)—C₁₋₆-alkylene-O;
with the proviso that L¹ and L² are linked to vicinal ring-members of the carbocycle;
R¹ is selected from the group consisting of —H; halogen; C₁₋₆ alkyl; C₂₋₆ alkenyl; C₂₋₆ alkynyl; —S—C₁₋₆ alkyl; C₁₋₆-haloalkyl; —O—C₁₋₆ haloalkyl; —S—C₁₋₆-haloalkyl; —OH; —SH; —CN; —NO₂ and —NR^{a}R^{b}, wherein R^{a} and R^{b} are independently H or C₁₋₆ alkyl;
R² is selected from the group consisting of —H; halogen; C₁₋₆ alkyl; C₂₋₆ alkenyl; C₂₋₆ alkynyl; —O—C₁₋₆ alkyl; —S—C₁₋₆ alkyl; C₁₋₆-haloalkyl; —O—C₁₋₆ haloalkyl; —S—C₁₋₆-haloalkyl; —OH; —SH; —CN; —NO₂ and —NR^{a}R^{b}, wherein R^{a} and R^{b} are independently H or C₁₋₆ alkyl;
with the proviso that at least one of R¹ and R² represents H;
R³, R⁴ independently are each selected from the group consisting of —H; halogen; C₁₋₆ alkyl; C₂₋₆ alkenyl; C₂₋₆ alkynyl; —O—C₁₋₆ alkyl; —S—C₁₋₆ alkyl; C₁₋₆-haloalkyl; —O—C₁₋₆ haloalkyl; —S—C₁₋₆-haloalkyl; —OH; —SH; —CN; —NO₂ and —NR^{a}R^{b}, wherein R^{a} and R^{b} are independently H or C₁₋₆ alkyl;
with the proviso that at least one of R³ and R⁴ does not represent H;
R⁵, R⁶ independently are each selected from the group consisting of —H; ═O; halogen; C₁₋₆ alkyl; C₂₋₆ alkenyl; C₂₋₆ alkynyl; —O—C₁₋₆ alkyl; —S—C₁₋₆ alkyl; C₁₋₆-haloalkyl; —O—C₁₋₆ haloalkyl; —S—C₁₋₆-haloalkyl; —OH; —SH; —CN; —NO₂ and —NR^{a}R^{b}, wherein R^{a} and R^{b} are independently H or C₁₋₆ alkyl.

2. A pharmaceutical composition according to claim 1 comprising one or more compounds of formula IA or a pharmaceutically acceptable salt thereof: 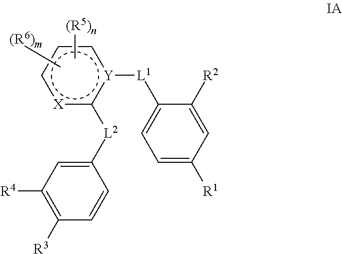
wherein
X, Y independently represent a carbon atom or a nitrogen atom; 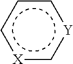
represents a 6-membered carbocycle, wherein said carbocycle is partially unsaturated or aromatic,
and
m, n, L¹, L², R¹, R², R³, R⁴, R⁵, R⁶ have the same meaning as defined in claim 1.

3. A pharmaceutical composition according to claim 1, comprising one or more compounds of formula IB or a pharmaceutically acceptable salt thereof: 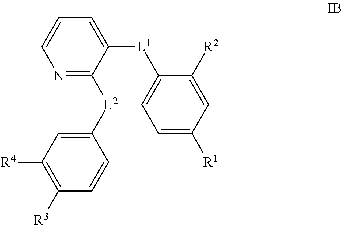
wherein L¹, L², R¹, R², R³ and R⁴ have the same meaning as defined in claim 1.

4. A pharmaceutical composition according to claim 3, comprising one or more compounds of formula IC or a pharmaceutically acceptable salt thereof: 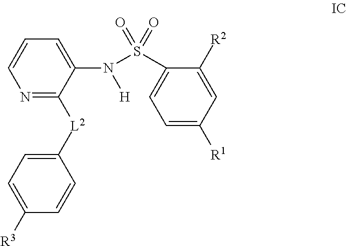
wherein
L² represents S or O;
R¹ is selected from the group consisting of —H; —F; —Cl; —Br; methyl; ethyl; n-propyl; iso-propyl; n-butyl; sec-butyl; iso-butyl; tert-butyl; —CF₃; —OCF₃; —SCF₃; —OH and —CN;
R² is selected from the group consisting of —H; —F; —Cl; —Br; methyl; ethyl; n-propyl; iso-propyl; n-butyl; sec-butyl; iso-butyl; tert-butyl; methoxy; ethoxy; —CF₃; —OCF₃; —SCF₃; —OH and —CN;
with the proviso that at least one of R¹ and R² represents H, and
R³ is selected from the group consisting of —F; —Cl; —Br; methyl; ethyl; n-propyl; iso-propyl; n-butyl; sec-butyl; iso-butyl; tert-butyl; methoxy; ethoxy; —CF₃, —OCF₃; —SCF₃; —OH and —CN.

5. A pharmaceutical composition according to claim 1, comprising one or more compounds of formula ID or a pharmaceutically acceptable salt thereof: 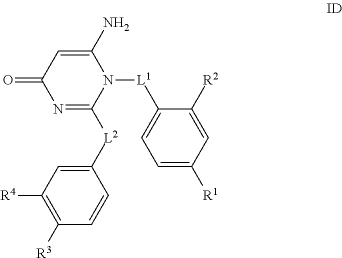
wherein L¹, L², R¹, R², R³ and R⁴ have the same meaning as defined in claim 1.

6. A pharmaceutical composition according to claim 5, comprising one or more compounds of formula IE or a pharmaceutically acceptable salt thereof: 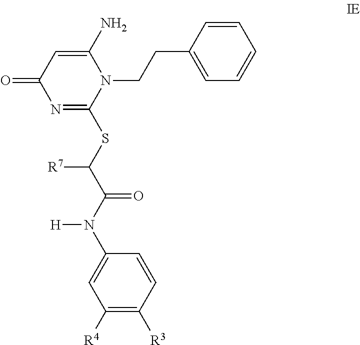
wherein
R⁷ is selected from the group consisting of H; methyl; ethyl; n-propyl; iso-propyl; n-butyl; iso-butyl; sec-butyl and tert-butyl;
R³, R⁴ are each selected from the group consisting of —H; —F, —Cl, —Br; methyl; ethyl; n-propyl; iso-propyl; n-butyl; sec-butyl; iso-butyl; tert-butyl; methoxy; ethoxy; —CF₃; —OCF₃; —SCF₃; —OH and —CN;
with the proviso that not both of R³ and R⁴ represent H.

7. A pharmaceutical composition according to claim 1, comprising one or more compounds selected from the group consisting of:
[1] N-(2-(4-chlorophenoxy)pyridin-3-yl)-4-isopropylbenzenesulfonamide,
[2] N-(2-(4-tert-butylphenoxy)pyridin-3-yl)-2-(trifluoromethyl)benzenesulfonamide,
[3] 4-chloro-N-(2-(p-tolylthio)pyridin-3-yl)benzenesulfonamide,
[4] 2-(6-amino-4-oxo-1-phenethyl-1,4-dihydropyrimidin-2-ylthio)-N-(3-chlorophenyl)propanamide and
[5] 2-(6-amino-4-oxo-1-phenethyl-1,4-dihydropyrimidin-2-ylthio)-N-(4-chlorophenyl)acetamide.

8. A method of treating a renal disorder in a patient in need of such treatment, said method comprising administering to said patient an effective amount therefor of a pharmaceutical composition according to claim 1.

9. Method according to claim 8, wherein the renal disorder is selected from the group consisting of kidney failure; podocyte damage; glomerular diseases; and diabetic nephropathy.

10. A method of treating cancer in a patient in need of such treatment, said method comprising administering to said patient an effective amount therefor of a pharmaceutical composition according to claim 1.

11. Method according to claim 10, wherein the cancer is selected from the group consisting of renal cancer, multiple myeloma, leukemia and colon cancer.

12. A method of treating a neurodegenerative disorder in a patient in need of such treatment, said method comprising administering to said patient an effective amount therefor of a pharmaceutical composition according to claim 1.

13. Method according to claim 12, wherein the neurodegenerative disorder is selected from the group consisting of Morbus Alzheimer, disorders associated with the deposition of beta-amyloid, age-related dementia, cerebral amyloidosis, systemic amyloidosis, hereditary cerebral hemorrhage with amyloidosis, Down's syndrome and ischemic stroke.

14. A method of treating a disorder in a patient in need of such treatment, said disorder being a disorder selected from the group consisting of disorders related to the eyes, kidneys, pancreas, prostate, mammae, liver, gall bladder, and mucosa, and said method comprising administering to said patient an effective amount therefor of a pharmaceutical composition according to claim 1.
